(19) United States
(12) Patent Application Publication
(10) Pub. No.: US 2012/0130155 A1
(43) Pub. Date: May 24, 2012

(54) DEVICE FOR THE TREATMENT OF FEMALE URINARY INCONTINENCE AND VAGINAL FLACCIDITY

(76) Inventor: José Bernardo Carvalho de Andrade, Macapa (BR)

(21) Appl. No.: 13/228,828

(22) Filed: Sep. 9, 2011

(30) Foreign Application Priority Data
Sep. 10, 2010 (BR) PI1004442-6

(51) Int. Cl. A61F 2/04

(52) U.S. Cl. 600/30

(57) ABSTRACT

A device for the treatment of female urinary incontinence and vaginal flaccidity including a silicon elastomer catheter of 10 cm in length×2.3 mm width and a rigid silicon connector in the shape of an hour glass that connects the extremities of the catheter to surgically treat female urinary incontinence and vaginal flaccidity.

### PRIORITY CLAIM

This patent application claims priority to Brazilian Patent Application No. PI1004442-6, filed Sep. 10, 2010, the disclosure of which is incorporated herein by reference in its entirety.

### FIELD

The present patent of invention has as objective the conception of a new device with the purpose of surgically treating female urinary incontinence, and still, vaginal flaccidity caused by labor trauma and/or aging. The advantages compared to the current surgical treatments are: the use of trans and post-op catheter will not be needed; trans-op cystoscopy will not be needed, indispensable in retropubic slings; there will be no need for anesthetic such as rachidian or peridural, since the anesthetic used is only local; there will be no need for hospital admittance; is less invasive; low cost; minimum post-op complications.

### BACKGROUND

Female urinary incontinence is the involuntary loss of urine through the urethra in improper situations, and its incidence in women increases with age, reaching 25% after menopause. The involuntary loss of urine acts in a devastating manner affecting the patient's quality of life, and it is estimated that there are over 30 million incontinent women in the USA alone.

The loss of urine can occur in a transitory manner, usually associated with the use of drugs, infections (urinary infections, vaginitis), constipation or hormonal deficiency problems, disappearing after treating the implied cause; or it can be persistent or definitive with settling and progressive worsening.

Many women become incontinent after labor, hysterectomy (surgery for the removal of the uterus) or even other trauma in the pelvic region or related neurological diseases.

Among the most common types of urine loss is the effort or stress urinary incontinence: characterized by the loss of urine when there is a sudden increase of intra-abdominal pressure like coughing, sneezing, laughing, jumping, running or making an effort.

The incontinence is diagnosed clinically, based on the detailed history of the patient, in which the beginning of the symptoms must be investigated, discarding the presence of urinary infection, stones, tumors, disease associated with mental illness, neuropathy and the use of medication. During the physical exam, the doctor must ask the patient to cough, trying to reproduce the urinary loss.

Also, a test can be done in which a cotton swab is inserted in the urethra to determine its position and mobility. An urine analysis exam must be made. An urodynamic test will determine if there are other alterations in the bladder and urethra.

Nowadays there are several products in the market with the purpose of treating urinary incontinence, the most used are called slings, among them are SPARC, MONARC, Safyre, TVT, Unitape tplus., Mini sling system (Ophira), etc. However, these products treat only the female urinary incontinence and the present invention treats, besides the urinary incontinence, the sexual dysfunction caused by vaginal flaccidity, and also providing future condition to place in the silicon catheter hormones of gradual liberation for therapeutic purposes.

### SUMMARY

To solve these problems, several researches and tests were conducted, and the one that presented a satisfactory result, was the “DEVICE FOR THE TREATMENT OF FEMALE URINARY INCONTINENCE AND VAGINAL FLACCIDITY”, reaching positive results in the treatment of urinary incontinence, and still, vaginal flaccidity, with greater efficiency, seen that the use of a trans and post-op catheter will not be needed; no need for trans-op cystoscopy, indispensible in retropubic slings; no need for anesthetic blocks, such as rachidian or peridural, since the anesthetic used is local; there will be no need for hospital admittance; is less invasive in comparison to the others; low cost; and presents minimum post-op complications.

### BRIEF DESCRIPTION OF THE DRAWING

FIG. 1 shows a side view of the device.

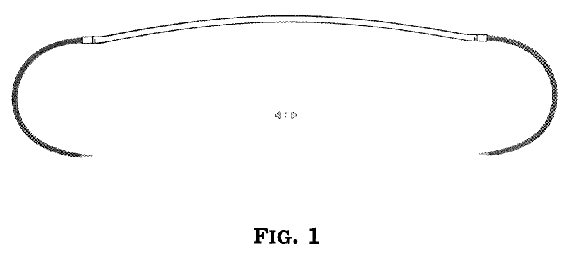

### DETAILED DESCRIPTION

The “DEVICE FOR THE TREATMENT OF FEMALE URINARY INCONTINENCE AND VAGINAL FLACCIDITY” is consisted of a silicon elastomer catheter of 10 cm in length×2.3 mm width and a rigid silicon connector in the shape of an hour glass that will have the function of connecting the extremities of the catheter, (see attached figure), with the purpose of surgically treating female urinary incontinence and vaginal flaccidity and in the future contain in the silicone catheter, slow release hormones with the objective of hormone replacement or contraceptive.

To manufacture the needles a steel thread of 2.5 mm in width was used. In a vice table the steel thread is fixed, in which a recess 1 mm in depth by 1.5 mm wide is made to fixate the extremity of the silicon catheter being implanted. Next, a semi-circle curvature is made to end up with a length of 8 cm. The other extremity is worn-out in a grinder to make a perforating extremity. These needles do not exist in the market and are made exclusively for the purpose at hand. The silicon catheter with the required characteristics does not exist in the market. They must contain the characteristics mentioned for length and width, and offer enough elasticity and consistency to provide support at the level of the urethra avoiding urinary loss and a soft constriction over the penis during sexual relations, not causing rupture of the ring nor exaggerated compression on the penis. Enough elasticity to enable the passage of vaginal speculum during gynecological exams.

For the implant of the “DEVICE FOR THE TREATMENT OF FEMALE URINARY INCONTINENCE AND VAGINAL FLACCIDITY”, it is recommended the following technique: local anesthetic with xylocaine at 1%; longitudinal incision from 1 cm to about 2 cm below the urethral meatus and another incision of the same length on the vaginal wall posterior to the level of the hymen remnant; introduction of one of the needles of the kit through the vaginal incision counter clock-wise, making a plication at the level of the right pubourethral/urethral pelvic ligament and follow with the needle until it emerges in the posterior vaginal incision. The other needle of the “DEVICE FOR THE TREATMENT OF FEMALE URINARY INCONTINENCE AND VAGINAL FLACCIDITY” is introduced in the same anterior vaginal incision and will make the trajectory in the opposite direction, also leaving in the posterior vaginal incision. The needles are disconnected and the extremities of the catheter are linked closing the ring. Next, the incisions are sutured. The estimated time to perform the surgery is from 25 to 30 minutes and after the procedure the patient is discharged home. The procedure is simple, less evasive, differing from the retropubic and transobturator slings regarding the fixation of the suburethral support, since in the recently created technique, the fixation is through the plication at the level of the pubourethral/urethral pelvic ligament and ischiocavernosus muscle under the mucous membrane, resulting in, besides the treatment of urinary incontinence, treat vaginal flaccidity as well, promoting the constriction of the vaginal canal.

In the present the “DEVICE FOR THE TREATMENT OF FEMALE URINARY INCONTINENCE AND VAGINAL FLACCIDITY” can be used in the surgical treatment of female urinary incontinence and sexual dysfunction, cause by vaginal flaccidity, and in the near future, it can be used as a substrate for hormonal substances with therapeutic purpose, similar to those already available in the market, such as the subcutaneous implants (norplant) and vaginal ring (NUVA RING) from Organon, containing hormones of gradual release as well as the intrauterine devices (IUD) containing hormones (progesterone) of gradual release (MIRENA).

1. The patent of invention of the “DEVICE FOR THE TREATMENT OF FEMALE URINARY INCONTINENCE AND VAGINAL FLACCIDITY” characterized for consisting in an elastomer silicon catheter of 10 cm in length×2.3 mm in width and a rigid silicon connector in the shape of an hourglass that will bind the extremities of the catheter, (see attached figure), with the purpose of surgically treating female urinary incontinence, vaginal flaccidity and later to be applied in the silicon catheter slow release hormones with the purpose of providing hormonal replacement or contraceptives.

2. The patent of invention of the “DEVICE FOR THE TREATMENT OF FEMALE URINARY INCONTINENCE AND VAGINAL FACIDEZ” characterized by having needles made by 2.5 mm thick steel threads. In a vice table the steel wire is fixed, in which a recess of 1 mm in depth by 1.5 mm wide to fix the extremity of the silicon catheter to be implanted. Next, a semi-circle curvature is made with a length of 8 cm. The other extremity is worn-out in a grinder until it becomes a perforating extremity. These needles do not exist in the market and are made exclusively for the proposed means. The silicon catheter with the required characteristics, do not exist in the market. The catheter must have the mentioned characteristics of length and width and offer enough elasticity and consistency to provide support at the level of the urethra avoiding urinary loss and soft constriction on the penis during intercourse, not causing disruption of the ring and exaggerated compression on the penis. Also, provide enough elasticity to enable passage of vaginal speculum during gynecological exams.

3. The patent of invention of the “DEVICE FOR THE TREATMENT OF FEMALE URINARY INCONTINENCE AND VAGINAL FLACCIDITY” characterized by its implant, in which the following technique is recommended: local anesthetic with xilocaine at 1%; longitudinal incision from 1 cm to about 2 cm under the urethral meatus and another incision of the same length in the posterior vaginal wall at the level of the hymen remnant; introduction of one of the needles of the kit through the vaginal incision counter clock-wise, making a plication at the level of the right pubourethral/urethral pelvic ligament and follow with the needle until it emerges in the posterior vaginal incision. The other needle of the “DEVICE FOR THE TREATMENT OF FEMALE URINARY INCONTINENCE AND VAGINAL FLACCIDITY” is introduced in the same anterior vaginal incision and will make the trajectory in the opposite direction, also leaving in the posterior vaginal incision. The needles are disconnected and the extremities of the catheter are linked closing the ring. Next, the incisions are sutured. The estimated time to perform the surgery is from 25 to 30 minutes and after the procedure the patient is discharged home. The procedure is simple, less evasive, differing from the retropubic and transobturator slings regarding the fixation of the suburethral support, since in the recently created technique, the fixation is through the plication at the level of the pubourethral/urethral pelvic ligament and ischiocavernosus muscle under the mucous membrane, resulting in, besides the treatment of urinary incontinence, treat vaginal flaccidity as well, promoting the constriction of the vaginal canal.
